(19) United States
(12) Patent Application Publication
(10) Pub. No.: US 2007/0038379 A1
(43) Pub. Date: Feb. 15, 2007

(54) Method and apparatus for predicting structure of transmembrane proteins

(76) Inventors: Nagarajan Vaidehi, Arcadin, CA (US); Wely B. Floriano, Pasadena, CA (US); Michael Singer, New Haven, CT (US); Gordon Shepherd, Hamden, CT (US); William A. Goddard III, Pasadena, CA (US)

Correspondence Address: FISH & RICHARDSON P.C., PO BOX 1022, MINNEAPOLIS, MN, 55440-1022, US

(21) Appl. No.: 09/816,755

(22) Filed: Mar. 23, 2001

Related U.S. Application Data
(60) Provisional application No. 60/191,896, filed on Mar. 23, 2000.
(60) Provisional application No. 60/213,659, filed on Jun. 23, 2000.

(51) Int. Cl. G06F 19/00; G01N 33/48; G01N 33/50

(52) U.S. Cl. 702/19

(57) ABSTRACT

Computer-implemented methods and apparatus implementing a hierarchical protocol using multiscale molecular dynamics and molecular modeling methods to predict the structure of transmembrane proteins such as G-Protein Coupled Receptors, and protein structural models generated according to the protocol. The protocol features a combination of coarse grain sampling methods, such as hydrophobicity analysis, followed by coarse grain molecular dynamics and atomic level molecular dynamics, including accurate continuum solvation, to provide a fast and accurate procedure for predicting GPCR tertiary structure.

### CROSS-REFERENCE TO RELATED APPLICATIONS

The present application claims benefit of U.S. Provisional Application No. 60/191,896, filed Mar. 23, 2000 and U.S. Provisional Application No. 60/213,659, filed Jun. 23, 2000, both of which are incorporated by reference herein.

### STATEMENT REGARDING FEDERALLY SPONSORED RESEARCH AND DEVELOPMENT

The U.S. Government has certain rights in this invention pursuant to Grant No. DAAG55-98-1-0266 awarded by the Department of the Army.

### REFERENCE TO AND INCORPORATION BY REFERENCE OF TABLES SUBMITTED ON COMPACT DISC

This application includes one or more tables (Tables 2 through 7) having over 50 pages of text which are submitted on compact disc. The material in question is contained in two (duplicate) compact discs, each of which includes the files Table2.txt (402 kilobytes), Table3.txt (406 kilobytes), Table4.txt (438 kilobytes), Table5.txt (390 kilobytes), Table6.txt (406 kilobytes) and Table7.txt (397 kilobytes), each of which was created on Mar. 23, 2001. All of the material in these files is incorporated by reference herein.

LENGTHY TABLES FILED ON CD
The patent application contains a lengthy table section. A copy of the table is available in electronic form from the USPTO web site (http://seqdata.uspto.gov/?pageRequest=docDetail&DocID=US20070038379A1) An electronic copy of the table will also be available from the USPTO upon request and payment of the fee set forth in 37 CFR 1.19(b)(3).

### BACKGROUND

The present invention relates to computational methods for predicting the three-dimensional structure of proteins and to computer-implemented apparatus for performing such computations. Proteins are linear polymers made up of 20 different naturally-occurring amino acids. The particular linear sequence of amino acid residues in a protein is said to define the protein's primary structure.

In its natural environment, a protein folds into a three-dimensional structure determined by its primary structure, and by the chemical and electronic interactions between the protein's individual amino acid constituents and the surrounding aqueous environment, which can include other biomolecules and cellular structures in addition to water. Studies of known three-dimensional structures have led to the identification of a number of characteristic patterns that appear to be particularly stable and therefore recur within folded proteins. Formed as a result of chemical interactions between different amino acids in the protein, these patterns, which include alpha helices, beta sheets and turns, among others, are referred to as the protein's secondary structure.

The combination of helices, sheets, turns and other structures that make up the protein's secondary structure, and the interactions between those structures, determine the protein's tertiary (or three-dimensional) structure. Because a protein's biological properties depend directly on its tertiary structure (i.e., its three-dimensional conformation), understanding that structure is crucial to understanding a protein's biological role and to applying that understanding in such areas as the treatment of disease and the design and development of new pharmaceuticals.

A protein's primary structure can be easily determined using known methods—for example, by identifying the amino acids coded for by a protein's known genetic sequence. Similarly, known techniques make it relatively easy to identify a protein's secondary structure once the primary structure is determined.

Determining a protein's tertiary structure is more difficult. For some proteins, it is possible to determine tertiary structure through such techniques as x-ray crystallography or spectroscopic methods such as fluorescence and nuclear magnetic resonance studies. However, these techniques can be time consuming and expensive, and not all proteins are equally amenable to structural examination by these methods.

One such class of proteins is the class of transmembrane proteins. These proteins—in particular the G-Protein Coupled Receptors (GPCR's)—are often involved in important cell recognition and communication processes. Indeed many diseases involve malfunctions of these proteins and many bacteria and viruses recognize particular GPCR's.

GPCR's share a predicted seven-transmembrane helix structure and the ability to activate a G-protein in response to ligand binding. Their natural ligands range from peptide and non-peptide neurotransmitters, hormones, and growth factors to odorants and light. The members of the GPCR superfamily which act through heterotrimeric G-proteins have been classified into six clans, as set out in Table 1.

TABLE 1
Classification of G-Protein Coupled Receptors

| | |
|---|---|
| 1. | Clan A: rhodopsin like receptors |

| | |
|---|---|
| Family I: | Olfactory receptors, adenosine receptors, |
| | melanocortin receptors and others. |
| Family II: | Biogenic amine receptors. |
| Family III: | Vertebrate opsins and neuropeptide receptors. |
| Family IV: | Invertebrate opsins. |
| Family V: | Chemokine, chemotactic, somatostatin, opioids and |
| | others. |
| Family VI: | Melatonin receptors and others. |

| | |
|---|---|
| 2. | Clan B: calcitonin and related receptors |

| | |
|---|---|
| Family I: | Calcitonin, calcitonin-like, and CRF receptors. |
| Family II: | PTH/PTHrP receptors. |
| Family III: | Glucagon, secretin receptors and others. |
| Family IV: | Latrotoxin receptors and others. |

| | |
|---|---|
| 3. | Clan C: metabotropic glutamate and related receptors |

Family I - Metabotropic glutamate receptors
Family II - Calcium receptors
Family III - GABA-B receptors
Family IV - Putative pheromone receptors

| | |
|---|---|
| 4. | Clan D: STE2 pheromone receptors |
| 5. | Clan E: STE3 pheromone receptors |

| | |
|---|---|
| 6. | Clan F: cAMP receptors |

One group of members of family I are the olfactory (odor) receptors (ORs) in the mammalian olfaction system. These receptors, unlike many other GPCRs that are designed for the specific recognition of few ligands, exhibit a combinatorial response to thousands of odorant molecules. Malnic, B., et al. (1999) Cell 96, 713-723. A single odor elicits response from multiple receptors and a single receptor also responds to multiple odorants, so every odorant has been thought to have a unique combination of responses from several receptors. Odor detection is mediated by approximately 1,000 ORs that are G protein coupled membrane-bound proteins. Malnic et al. recently reported the differential responses of individual mouse OR neurons to 24 organic odor compounds (linear alcohols, acids, diacids, and bromoacids with four to nine carbons) by using Ca²⁺-imaging techniques, followed by single-cell reverse transcription-PCR to determine the sequence of the responsive OR. These results lead to the compelling question “what is the molecular basis of odor recognition?” Such questions can be answered only with an understanding of the atomic-level structure of these ORs.

Extensive protein sequence analyses on the class of GPCR's has revealed a common topology consisting of a membrane-spanning seven-helix bundle, as discussed above, which is believed to accommodate the binding site for low-molecular weight ligands. However, although much effort has been put into elucidating the structure of GPCRs, only a very small number of complete 3D structures of transmembrane proteins are known from experiments (e.g., bacteriorhodopsin and bovine rhodopsin).

As a result, there is a need for modeling techniques that predict structure and functional characteristics of the members of this class of proteins at a molecular level.

### SUMMARY OF THE INVENTION

The invention provides a hierarchical protocol using multiscale molecular dynamics and molecular modeling methods to predict the structure of G-Protein Coupled Receptors. The protocol features a combination of coarse grain sampling methods, such as hydrophobicity analysis, followed by coarse grain molecular dynamics and atomic level molecular dynamics, including accurate continuum solvation, to provide a fast and accurate procedure for predicting GPCR tertiary structure.

In general, in one aspect, the invention features methods and apparatus, including computer program apparatus, implementing techniques for predicting the structure of a membrane-bound protein having a plurality of α-helical regions. The techniques can include providing an amino acid sequence for the membrane-bound protein; using the amino acid sequence to identify one or more transmembrane regions of the membrane-bound protein; constructing a set of helices for the transmembrane regions and optimizing a helix bundle configuration for the set of helices using a first molecular dynamics simulation; constructing a plurality of inter-helical loops to generate a full-atom model of the membrane-bound protein; optimizing the full-atom model using a second molecular dynamics simulation; and outputting a predicted structure for the transmembrane protein based on the second optimization. In particular implementations, constructing the set of helices for the transmembrane regions can include constructing a set of canonical helices corresponding to the transmembrane regions, calculating a minimum-energy configuration for each of the canonical helices, optimizing each of the canonical helices, assembling a helix bundle including each of the set of helices, and calculating a minimum-energy configuration for the helix bundle in a lipid bilayer.

In general, in another aspect, the invention features additional methods and apparatus, including computer program apparatus, implementing techniques for modeling the structure of a transmembrane protein having a plurality of α-helical regions. The techniques can include providing amino acid sequence information and sequence alignment information for a transmembrane protein having a plurality of α-helical regions; using the amino acid sequence information and the sequence alignment information to predict a set of transmembrane segments of the transmembrane protein; constructing canonical helices for the predicted transmembrane segments and optimizing the canonical helices using a first molecular dynamics simulation; combining the optimized helices based on the sequence alignment information to form a helix bundle, and assembling the helix bundle with a lipid bilayer to form a system helix bundle; optimizing the structure of the system helix bundle using a second molecular dynamics simulation; adding inter-helical loops to the system helix bundle to form a full atom model; optimizing the full atom model using a third molecular dynamics simulation; and outputting a predicted structure for the transmembrane protein based on the third optimization.

Particular implementations of the invention can include one or more of the following features. The transmembrane protein can be a G-protein coupled receptor. A energy minimum can be calculated for each of the canonical helices before forming the helix bundle. The techniques can also include determining the periodicity of hydrophobic residues identified in the amino acid sequence information; and identifying a plurality of lipid-accessible residues based at least in part on the identified periodicity. The helix axes can be oriented according to the 7.5 Å electron density map for rhodopsin. The identified lipid-accessible residues can be oriented to face the outside of the helix bundle. The first molecular dynamics simulation can include torsional molecular dynamics simulations, such as Newton-Euler Inverse Mass Operator molecular dynamics. The second molecular dynamics simulation can include rigid body molecular dynamics simulations. The third molecular dynamics can include mixed mode molecular dynamics simulations. At least the third simulation can preferably include solvent approximations, such as a continuum solvation model or empirical solvation model based on estimating salvation free energy based on solvent-accessible protein surface area. Some examples of appropriate solvent models include the Surface Generalized Born model or a Poisson-Boltzmann description model. The predicted structure can be generated by performing the third molecular dynamics simulation for a time in the range from about 100 ps to about 1 ns.

In general, in another aspect, the invention features computational models of the structure of transmembrane proteins having a plurality of α-helical regions. The computational models can include computer-readable data storage media storing data describing a predicted three-dimensional structure for such proteins, including, for example, olfactory receptors S6, S18, S19, S25, S46 or S50. The data describing the three-dimensional protein structure can describe optimized predicted structures generated according to the techniques recited above.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating a general seven-helical membrane-bound protein.

FIG. 2 is a block diagram illustrating a general protocol for predicting GPCR structure according to the invention.

FIG. 3 is a block diagram illustrating a modeling system suitable for performing the structure prediction protocols of the invention.

FIG. 4 is a flow diagram illustrating a particular implementation of the protocol of FIG. 2.

FIG. 5 shows the structures predicted for bacteriorhodopsin using the protocol of FIG. 4.

FIG. 6 shows the predicted structures for six mouse olfactory receptors generated according to the protocol of FIG. 4.

### DETAILED DESCRIPTION

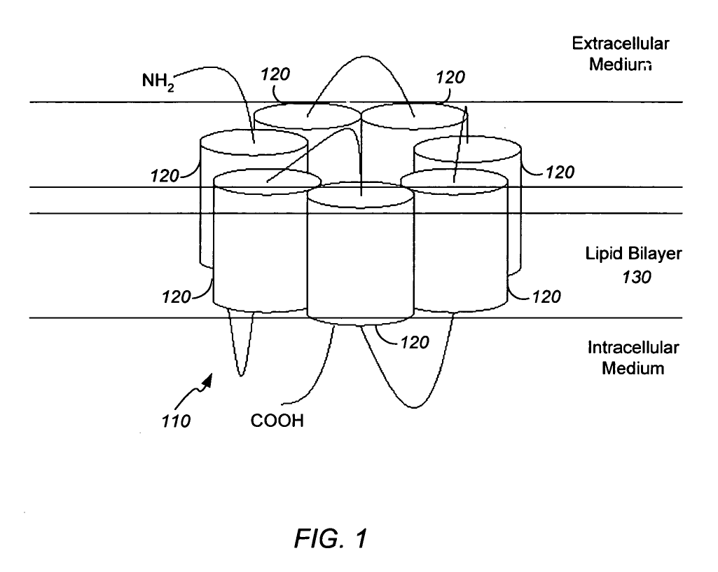

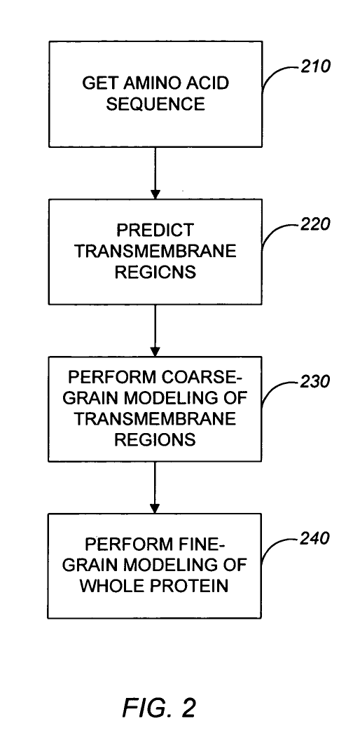

The present invention provides a computational hierarchical strategy for predicting the structure of certain transmembrane proteins such as G-protein coupled receptor 110, illustrated in FIG. 1 as a bundle of seven helices 120 embedded in membrane 130. In brief, as illustrated in FIG. 2, starting with the amino acid sequence for protein 110 (obtained in step 210 from the commercial GeneBank database, for example), the protein structure prediction strategy 200 predicts the transmembrane helical domains for protein 110 (step 220). The method then performs coarse grain modeling of the predicted transmembrane regions (step 230). The method concludes with fine grain modeling of the whole protein (step 240) to yield a three-dimensional model of transmembrane protein 110,

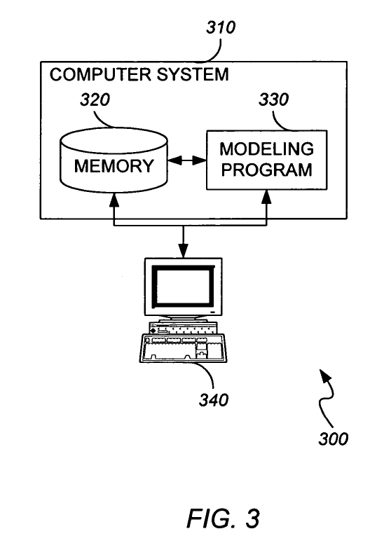

The techniques described herein can be implemented using a modeling system 300 as shown in FIG. 3. Modeling system 300 includes a general-purpose programmable digital computer system 310 of conventional construction, including a memory 320 and a processor for running a modeling program or programs 330. Modeling system 300 also includes input/output devices 340, and, optionally, conventional communications hardware and software by which computer system 310 can be connected to other computer systems. Although FIG. 3 illustrates modeling system 300 as being implemented on a single computer system, the functions of system 300 can be distributed across multiple computer systems, such as on a network. Those skilled in the art will recognize that system 300 can be implemented in a variety of ways using known computer hardware and software, such as, for example, a Silicon Graphics Origin 2000 server having multiple R10000 processors running at 195 MHz, each having 4 MB secondary cache, or a dual processor Dell PowerEdge system equipped with Intel PentiumIII 866 MHz processors with 1 Gb of memory and a 133 MHz front side bus.

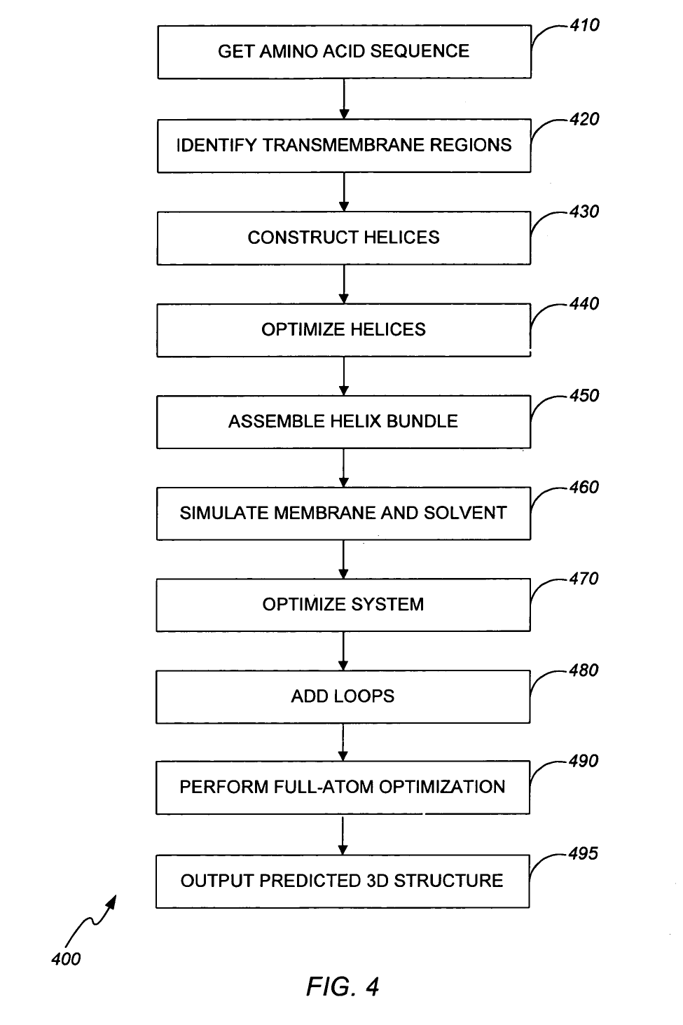

A particular implementation of the protocol described in FIG. 2 will now be described in more detail. As illustrated in FIG. 4, the method 400 starts with an amino acid sequence obtained from memory 320 or some other source, such as a commercial database as discussed above (step 410). The sequence information is used to identify transmembrane regions (step 420). In one implementation, transmembrane regions are identified on the basis of amino acid hydrophobicity using the multisequence profile method of Donnelly, D. (1993) Biochem. Soc. Trans. 21, 36-39 (implemented, e.g., in PERSCAN), which is incorporated by reference herein. Sequences are aligned by the iterative profile alignment utility of WHATIF, according to Vriend, G. (1990) J. Mol. Graphics 8, 52-56, which is incorporated by reference herein. The output from this step is a range or ranges of amino acids in the sequence that are predicted to be in the transmembrane region.

This range information is used to construct canonical right-handed α-helices (step 430) using known secondary structure modeling techniques, such as Polygraf, Builder and/or Homology software applications of Molecular Simulations, Inc., of San Diego, Calif. These helices are then optimized (step 440), e.g., using the Newton-Euler Inverse Mass Operator torsional MD method as described in Jain, A. et al. (1993) J. Comp. Phys. 106, 258-268; Mathiowetz, A. M., et al. (1994) Proteins 20, 227-247; and Vaidehi, N., et al. (1996) J. Phys. Chem. 100, 10508-10517, each of which is incorporated by reference herein. The output from this optimization step is a set of 3-D coordinates for the final optimized structure for each helix.

To assemble the helix bundle, helical rotations and the orientation of each helical axis are predicted (step 450)—for example, using the bovine rhodopsin 7.5-Å electron density map, according to Schertler, G. F. X. (1988) Eye 12, 504-510, which is hereby incorporated herein—to obtain x and y coordinates as well as tilt for each helix. The helical axes orientation can also be incorporated from the 2.8 Å structure of Palczewski et al (2000) Science, 289, 739-745. In one implementation, helical z-coordinates are set such that the midpoint of each helical axis is positioned in the same z-plane of the assembly. Likewise, lipid-accessible residues, identified from sequence alignments and from analysis of the periodicity of hydrophobic residues in the sequence, can be oriented to face the outside of the helix barrel. To further optimize the packing of the helix bundle, the effect of the environment of protein 310 is simulated with a continuum description of the water environment and a lipid bilayer to simulate membrane 320 (step 460).

This combined system is optimized using rigid body dynamics (step 470)—e.g., using the DREIDING force field with polar group charges derived from charge equilibration to simulate the lipids and charges from CHARMM22 for the protein, according to Mayo, S. L., et al. (1990) J. Phys. Chem. 94, 8897-8909, Rappe, A. K., et al. (1991) J. Phys. Chem. 95, 3358-3363, and MacKerell, A. D., et al. (1998) J. Phys. Chem. B 102, 3586-4616, each of which is incorporated by reference herein. In one experiment (Example 2, below), the performance of this combination of charges and parameters was evaluated through a series of constant temperature and pressure MD simulations of crystals. The systems 1,2-dilauroyl-DL-phosphatidyl ethanolamine acetic acid, disodium β-glycerophosphate hydrate, and L-α-glycerol phosphorylcholine were chosen for simulation to evaluate the performance of the force field and atomic charges progressively, from a simple polar head group to a crystal lipid bilayer. Comparison of the results of these simulations with experimental data and other simulation results available in the literature showed that the choice of charges and force field gives densities and cell parameters with less than 4% error from the experimentally determined parameters. The rigid body dynamics was done for 150 ps by which time equilibration was attained.

Loops are then added to the helices (step 480), using known techniques. In one implementation, loops are added using the WHATIF software referred to above, although any comparable loop-building software, including commercially available software could be used. After the addition of loops, a full atom minimization of the complete model with a barrel of lipid surrounding the protein is performed, followed by dynamic optimization of the structure by using the Massively Parallel Simulation program (MPSim) (step 490), according to Lim, K. T., et al., J. Comput. Chem. 18, 501-521 (1997), which is incorporated by reference herein. The MPSim software implements molecular dynamics techniques such as: (i) the cell multipole method for fast and accurate calculation of nonbond forces, according to Ding, H. Q. et al. (1992) J. Chem. Phys. 97, 4309-4315, and Ding, H. Q., et al. (1992) Chem. Phys. Lett. 196, 6-10, both of which are incorporated by reference herein; (ii) fast torsional dynamic methods such as Newton-Euler Inverse Mass Operator, according to Jain, A. et al. (1993) J. Comp. Physiol. 106, 258-268; Mathiowetz, A. M., et al. (1994) Proteins 20, 227-247; and Vaidehi, N., et al. (1996) J. Phys. Chem. 100, 10508-10517, incorporated by reference above, and Hierarchical Newton-Euler Inverse Mass Operator, according to Vaidehi, N., et al. (2000) J. Phys. Chem. 104, 2375-2383, which is incorporated by reference herein; (iii) continuum solvation techniques such as the Poisson-Boltzmann description, according to Tannor, D. J., et al. (1994) J. Am. Chem. Soc. 116, 11875-11882, which is incorporated by reference herein, and surface-generalized Born model that account for solvation in biological systems, according to Ghosh, A., et al. (1998) J. Phys. Chem B. 102, n 10983-10990, which is incorporated by reference herein. Those skilled in the art will recognize that other solvation models can be used—for example, empirical solvation models that estimate solvation free energies as a function of solvent accessible surface area of the protein, as described in Williams, R. L., et al. (1992) Proteins: Structure, Function and Genetics 14, 110-119, which is incorporated by reference herein.

In one implementation, the solution structure is optimized by performing mixed mode dynamics using the following descriptions. The helices and loops in the protein are modeled with the Newton-Euler Inverse Mass Operator torsional molecular dynamics. The lipids are treated as rigid bodies, and the counterions Na⁺ and Cl⁻ as free Cartesian atoms. Constant temperature dynamics using the Hoover algorithm is performed for 50 ps with time steps of 1 and 5 fs. The outside of the lipid layer is simulated with surface-generalized Born model continuum solvent description. A low dielectric constant of 60.0 is used to simulate the low dielectric region surrounding the membrane.

Those skilled in the art will recognize that the decision when to terminate the molecular dynamics optimization (and thereby the decision whether a particular calculated structure is a “final” structure for the purposes of the following step fo the method) is to some extent up to the user, and for particular implementations may depend on such factors as computing power, time and the degree of precision desired in the predicted results. The results of the molecular dynamics simulations can be considered to comprise a series of snapshots, taken as the dynamics simulation progresses. While in many cases it may be desirable to allow the simulations to proceed until they reach equilibrium (e.g., the point at which additional processing time no longer produces significant changes in the calculated optimized structure), that need not always be the case. Accordingly, the particular duration of the molecular dynamics optimization steps is not critical to the methods disclosed herein. In some preferred implementations, simulations are run for times in the range from about 100 ps to about 1 ns, and the structures produced by the method can include any individual snapshots taken within these time constraints.

Following the final optimization of step 490, the method outputs a final predicted 3-D structure of the entire protein and surrounding lipids (step 495), which may be generated in a standard format such as the protein data bank (pdb) format. As discussed above, the characterization of a particular predicted structure as a “final” structure will depend on the user's determination of the appropriate duration of the optimization of step 490. Depending on which individual snapshot is chosen as the “final” structure, the particular predicted atomic coordinates may differ, as a result of additional optimization. Accordingly, those skilled in the art will recognize that the output structures generated in step 495 may differ even for a given set of input parameters. In particular implementations, these differences can be expected to include differences in root mean square deviation of the predicted coordinates for atoms in the protein's amide backbone of less than or equal to about 2.0 Å.

The techniques and apparatus described herein may have useful application in the modeling of any transmembrane protein having one or more membrane-spanning α-helices, where the protein's primary structure (amino acid sequence) is known and for which an experimental or theoretical helical template is available. In particularly advantageous implementations, the techniques and apparatus are useful in modeling the structure of having a relatively large number (e.g., about 4 or more) membrane-spanning α-helical regions, such as the seven-helical GPCR's described in Example 2, below.

The output structures can be used in further studies, including, for example, ligand docking studies directed to modeling receptor binding sites, for the purpose of identifying natural or synthetic receptor ligands, or for developing synthetic receptors that exhibit behavior analogous to naturally-occurring GPCR's. The predicted structures can also be useful in identifying regions of cellular receptors that bind microbial pathogens. Subsequently using this model of the first step of pathogenesis one could design competitive inhibitors either on the receptor or for the microbial surface structures.

### EXAMPLE 1

The protocol described above was tested on bacteriorhodopsin (BRDP), a membrane protein for which the crystal structure has been fitted with fair accuracy in the transmembrane region of the protein. Starting from the sequence of bacteriorhodopsin, and without using coordinates from the crystal structure, the protocol described above was used to build the complete protein model. The membrane was represented by bilayers of diphosphatidyl glycerophosphate that is the lipid present in the purple membrane from Halobacterium halobium. Although the sequence homology between BRDP and the ORs is not high (less than 30%), they share the same tertiary motif common to α-helical transmembrane proteins: a 7-helix barrel.

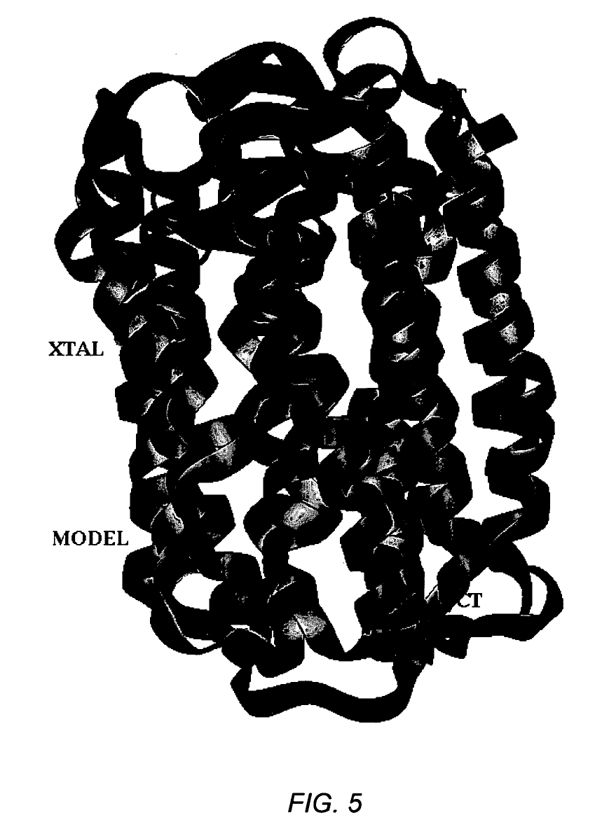

As shown in FIG. 5, the predicted tertiary structure for BDRP compares favorably with the known crystal structure. The overall rms deviation in coordinates of C_{α} atoms from the crystal structure for the final model is 5.98 Å for all 221 aa. The overall rms deviation in coordinates for the residues in the membrane region is 3.29 Å whereas that for the loops is 8.57 Å. Thus, the modeling procedure described above gives a reasonable structure as compared with the crystal structure for a known membrane protein.

### EXAMPLE 2

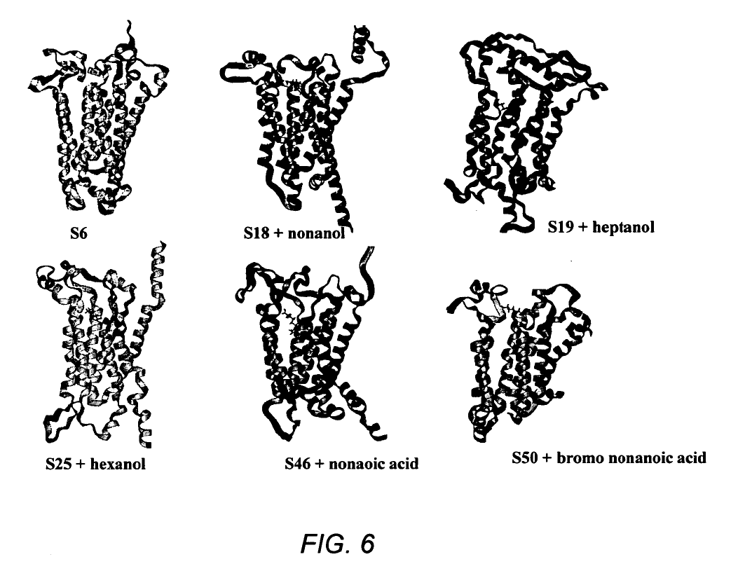

Modeling of six olfactory receptors. Sequences for ORS25, ORS18, ORS19, ORS6, ORS46 and ORS50 were obtained from taken from Malnic et al. For each receptor, the membrane was simulated by using explicit lipid bilayers of dilauroylphosphatidyl choline. The choice of lipid in the OR case is supported by experimental indications, Gimenez, C. (1998) Rev. Neurol. (Paris) 26, 232-239; Kiefer, H. et al. (1996) Biochemistry 35, 16077-16084, that the membrane surrounding the ORs in vivo can be satisfactorily simulated by using a single-component lipid system of dilauroylphosphatidyl choline. Final atomic level models for the six receptors are shown in FIG. 6. Predicted structural models for S6, S18, S19, S25, S46 and S50 are included in PDB format in Tables 2 through 7, respectively, submitted on compact disc and incorporated by reference above. An explanation of the PDB file format can be found at http://www.rcsb.org/pdb/. See also Berman, H. M., et al. (2000) Nucleic Acids Res. 28, 235-242.

The final atomic level model of one of these receptors—olfactory receptor S25—was used in docking studies as described in U.S. Provisional Application No. 60/213,658, filed Jun. 23, 2000, and Floriano, W. B., et al. (2000) PNAS 97, 10712-10716, both of which are incorporated by reference herein. As described in those references, calculated binding energies for a series of alcohols, carboxylic acids, dicarboxylic acids and bromocarboxylic acids containing 4-9 carbon atoms showed good correlation to the measured recognition profiles for the two known odorants of OR S25 (hexanol and heptanol).

The invention can be implemented in digital electronic circuitry, or in computer hardware, firmware, software, or in combinations of them. Apparatus of the invention can be implemented in one or more computer program products tangibly embodied in a machine-readable storage device for execution by a programmable processor; and method steps of the invention can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

A number of implementations of the invention have been described. Nevertheless, it will be understood that various modifications may be made without departing from the spirit and scope of the invention. Accordingly, other embodiments are within the scope of the following claims.

1. A computer implemented method for predicting the structure of a membrane-bound protein having a plurality of α helical regions, comprising:
providing an amino acid sequence for the membrane-bound protein;
identifying a range of amino acids in the amino acid sequence as transmembrane regions of the membrane-bound protein;
constructing each of two or more helices in a set of helices for the transmembrane regions;
optimizing a helix bundle configuration for the set of helices using a first molecular dynamics simulation;
after optimizing the helix bundle configuration, constructing one or more inter-helical loops to generate a full-atom model of the membrane-bound protein;
optimizing the full-atom model using a second molecular dynamics simulation; and
outputting a predicted structure for the membrane-bound protein based on the second optimization.

2. (canceled)

3. The method of claim 1, wherein:
constructing each of two or more helices in the set of helices for the transmembrane regions includes constructing each of two or more canonical helices corresponding to the transmembrane regions, calculating a minimum-energy configuration for each of the canonical helices, and optimizing each of the canonical helices.

4-34. (canceled)

35. The method of claim 3, wherein:
optimizing a helix bundle configuration includes calculating a minimum-energy configuration for the helix bundle in a lipid bilayer.

36. The method of claim 1, wherein:
the membrane-bound protein is a G-protein coupled receptor.

37. The method of claim 1, wherein:
identifying a range of amino acids in the amino acid sequence as transmembrane regions includes aligning the amino acid sequence with an experimental or theoretical helical template.

38. The method of claim 1, wherein:
identifying a range of amino acids in the amino acid sequence as transmembrane regions includes determining the periodicity of hydrophobic residues in the amino acid sequence; and
optimizing a helix bundle configuration includes identifying a plurality of lipid-accessible residues based at least in part on the determined periodicity.

39. The method of claim 1, wherein:
constructing each of two or more helices in a set of helices for the transmembrane regions includes optimizing each of the two or more helices in the set of helices using a torsional molecular dynamics method.

40. The method of claim 39, wherein:
the torsional molecular dynamics method uses the Newton-Euler Inverse Mass Operator.

41. The method of claim 1, wherein:
constructing each of two or more helices in a set of helices for the transmembrane regions includes determining 3-D coordinates that define the structure of each helix in the set of helices.

42. The method of claim 1, wherein:
optimizing a helix bundle configuration includes determining a rotation and tilt of each helix in the set of helices.

43. The method of claim 1, wherein:
optimizing a helix bundle configuration includes orienting the helix axes according to the 7.5 Å electron density map for rhodopsin.

44. The method of claim 38, wherein:
optimizing a helix bundle configuration includes orienting the identified lipid-accessible residues to face the outside of the helix bundle.

45. The method of claim 1, wherein:
the first molecular dynamics simulation is a rigid body molecular dynamics simulation.

46. The method of claim 1, wherein:
optimizing a helix bundle configuration for the set of helices includes modeling an effect of an environment of the membrane-bound protein, wherein the effect of the environment is simulated with a continuum description of a water environment and a lipid bilayer.

47. The method of claim 45, wherein:
the first molecular dynamics simulation uses the DREIDING force field, charges simulating the membrane, and charges for the membrane-bound protein.

48. The method of claim 1, wherein:
the second molecular dynamics simulation is a mixed mode molecular dynamics simulation.

49. The method of claim 48, wherein:
the second molecular dynamics simulation uses a torsional molecular dynamics method to model the helices and inter-helical loops and a rigid body molecular dynamics method to model the membrane of the membrane-bound protein.

50. The method of claim 1, wherein:
the second molecular dynamics simulation includes dynamic optimization of the structure using cell multipole methods or fast torsional dynamic methods.

51. The method of claim 1, wherein:
at least the second molecular dynamics simulation includes a solvent approximation.

52. The method of claim 51, wherein:
the solvent approximation is a continuum solvation model.

53. The method of claim 52, wherein:
the solvent approximation includes the Surface Generalized Born model or the Poisson-Boltzmann description.

54. The method of claim 53, wherein:
the solvent approximation is an empirical approximation comprising estimating solvation free energy as a function of solvent accessible protein surface area.

55. The method of claim 1, wherein:
the predicted structure is generated by performing the second molecular dynamics simulation for a time in the range from about 100 ps to about 1 ns.

56. The method of claim 1, wherein:
the set of helices includes four or more membrane-spanning α-helices.

57. The method of claim 1, wherein:
the set of helices includes seven membrane-spanning α-helices.

58. The method of claim 1, wherein said identifying comprises identifying ranges of amino acids in the amino acid sequence as transmembrane regions of the membrane-bound protein.
